(19) United States
(12) Patent Application Publication
(10) Pub. No.: US 2010/0317954 A1
(43) Pub. Date: Dec. 16, 2010

(54) CONDUCTIVE ELASTIC BAND

(75) Inventors: Yeonhee Jeong, Daejeon (KR); Young-Ju Jeon, Daejeon (KR); Seungchul Shin, Daejeon (KR); Seunghwan Kim, Daejeon (KR)

Correspondence Address: LAHIVE & COCKFIELD, LLP;FLOOR 30, SUITE 3000, ONE POST OFFICE SQUARE, BOSTON, MA, 02109, US

(73) Assignee: Electronics and Tlecommunications Research Institute, Daejeon (KR)

(21) Appl. No.: 12/517,960

(22) PCT Filed: Dec. 3, 2007
(86) PCT No.: PCT/KR2007/006172

§ 371 Date: Jun. 5, 2009

(30) Foreign Application Priority Data
Dec. 7, 2006 (KR) 10-2006-0124112

(51) Int. Cl. A61B 5/04

(52) U.S. Cl. 600/372

(57) ABSTRACT

Provided is a conductive elastic band that can obtain vital information in smart clothes, wearable computers, or bio-shirts, can transfer the obtained vital information externally through a communication network, and can be applied to various kinds of fabrics and clothes while maintaining conductivity and flexibility in shape. The conductive elastic band includes a front surface in contact with the user including a conductive portion having a fabric electrode and a non-conductive portion weaved with general fibers, a non-conductive rear surface weaved with the general fibers and elastic warps located between the front surface and the rear surface.

### TECHNICAL FIELD

The present invention relates to a conductive elastic band, and more particularly, an elastic band weaved with a conductive thread which can obtain vital information through smart clothing, wearable computers, or bio-shirts, can transfer the vital information externally using a communication network, can be applied to various materials and clothing, and can be structurally manipulated while maintaining conductivity. This work was supported by the IT R&D program of MIC/IITA [2006-S-007-01, ubiquitous health monitoring module and system development].

### BACKGROUND ART

Conventionally, in order to obtain vital information, a disposable vital information sensor is used or industrial conductive fabric is used. However, it is difficult to obtain the vital information stably and comfortably using the above disposable vital information sensor or the industrial conductive fabric. The disposable vital information sensor can only be used for a short period of time if there is any body movement, and thus, it is difficult to obtain the vital information. The industrial conductive fabric, although having superior conductivity, is uncomfortable to wear.

For example, in order to obtain vital information such as an electrocardiogram, it is important for the sensor to be attached to the human body. A disposable vital information sensor presses against the human body using a gel which can lead to discomfort. Industrial conductive fabric is not flexible and cannot be attached to the human body at all. In addition, industrial conductive thread and wires do not have any elasticity, and it is difficult to fix the industrial conductive thread or the wires in clothes. Therefore, use of either method may leave the user feeling uncomfortable and unable to move actively.

### DISCLOSURE OF INVENTION

### Technical Problem

The present invention provides a conductive elastic band including a front surface in contact with a user, the band including a conductive portion having a fabric electrode and a non-conductive portion weaved with general fibers, a non-conductive rear surface weaved with the general fibers and elastic warps located between the front surface and the rear surface.

### Technical Solution

The conductive elastic band of the present invention can form a fabric electrode that can obtain vital information while maintaining elasticity of the band by forming a surface (front surface) contacting portion from which the vital information is obtained, using conductive thread, and forming other portions using general fiber. The conductive elastic band is sewed with conductive elastic thread so that the vital information can be easily transferred through the thread. Also, the size of texture of the conductive elastic band can be freely changed according to functions and objects, and thus, the conductive elastic band can be used in various applications.

### ADVANTAGEOUS EFFECTS

According to the present invention, the area of the conductive sensor in the conductive elastic band can be freely adjusted according to the object of using the sensor, and the sensor can be stably attached to the human body due to the high elasticity while maintaining a level of comfort for the user. The information obtained using the conductive elastic band is transferred through the conductive elastic threads without interfering with the actions of the human body, and thus, the vital information can be obtained stably while increasing the freedom and the comfort of the user. Moreover, the conductive elastic band can obtain and transfer the vital information in all kinds of clothes such as functional sports wears, outdoor clothes, patient clothes, and specialized functional wears.

In particular, the conductive elastic band of the present invention is weaved using different kinds of fibers and structures on the front and rear surfaces so as to have the conductive surface and the non-conductive surface, and thus, it can be efficiently used to obtain the vital information of the user. In addition, the conductive elastic band can be washed, managed, and formed in various shapes in bio-clothes obtaining the vital information while maintaining the comfort of the user, and thus, can solve the problems of the conventional non-flexible wires or non-elastic conductive threads.

### DESCRIPTION OF DRAWINGS

The above and other features and advantages of the present invention will become more apparent by describing in detail exemplary embodiments thereof with reference to the attached drawings in which:

FIG. 1 is a front view of a conductive elastic band according to an embodiment of the present invention;

FIG. 2 is a rear view of the conductive elastic band of FIG. 1;

FIG. 3 is a simplified side view of the conductive elastic band of FIG. 1;

FIG. 4 is a detailed front view of structural elements in the conductive elastic band of FIG. 1;

FIG. 5 is a detailed rear view of structural elements in the conductive elastic band of FIG. 1;

FIG. 6 is a rear view showing structural elements of the conductive elastic band of FIG. 1;

FIG. 7 is a front view showing structural elements of the conductive elastic band of FIG. 1; and

FIG. 8 is a front view of clothing including the conductive elastic band of FIG. 1 and thread according to an embodiment of the present invention.

### BEST MODE

The conductive elastic band of the present invention can form a fabric electrode that can obtain vital information while maintaining elasticity of the band by forming a surface (front surface) contacting portion from which the vital information is obtained, using conductive thread, and forming other portions using general fiber. The conductive elastic band is sewed with conductive elastic thread so that the vital information can be easily transferred through the thread. Also, the size of texture of the conductive elastic band can be freely changed according to functions and objects, and thus, the conductive elastic band can be used in various applications.

### MODE FOR INVENTION

The present invention provides a conductive elastic band that can obtain vital information stably and continuously, and can be freely applied to clothing because it provides the user with comfort and freedom of movement.

The conductive elastic band of the present invention can form a fabric electrode that can obtain vital information while maintaining elasticity of the band by forming a surface (front surface) contacting portion from which the vital information is obtained, using conductive thread, and forming other portions using general fiber. The conductive elastic band is sewed with conductive elastic thread so that the vital information can be easily transferred through the thread. Also, the size of texture of the conductive elastic band can be freely changed according to functions and objects, and thus, the conductive elastic band can be used in various applications.

Hereinafter, structures and operation of the invention according to embodiments of the present invention will be described in detail with reference to accompanying drawings. Like reference numerals in the drawings denote like elements.

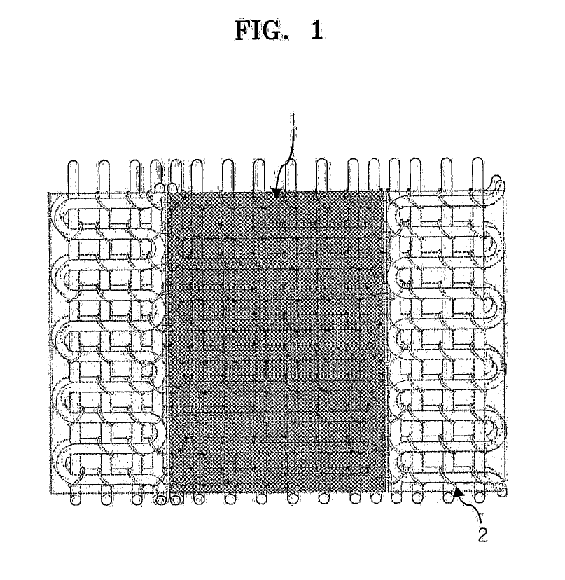

FIG. 1 is a front view of a conductive elastic band according to an embodiment of the present invention.

A front surface is a surface contacting the user. Referring to FIG. 1, a fabric electrode, that is, a conductive portion 1 is weaved using a conductive thread, and a non-conductive portion 2 is weaved using a general fiber which can be sewed in various ways. The fabric electrode 1 can obtain the user's vital information. The width and shape of the fabric electrode 1 can vary with the shape of cloth, the vital information measurement portion, and the measuring method of vital information.

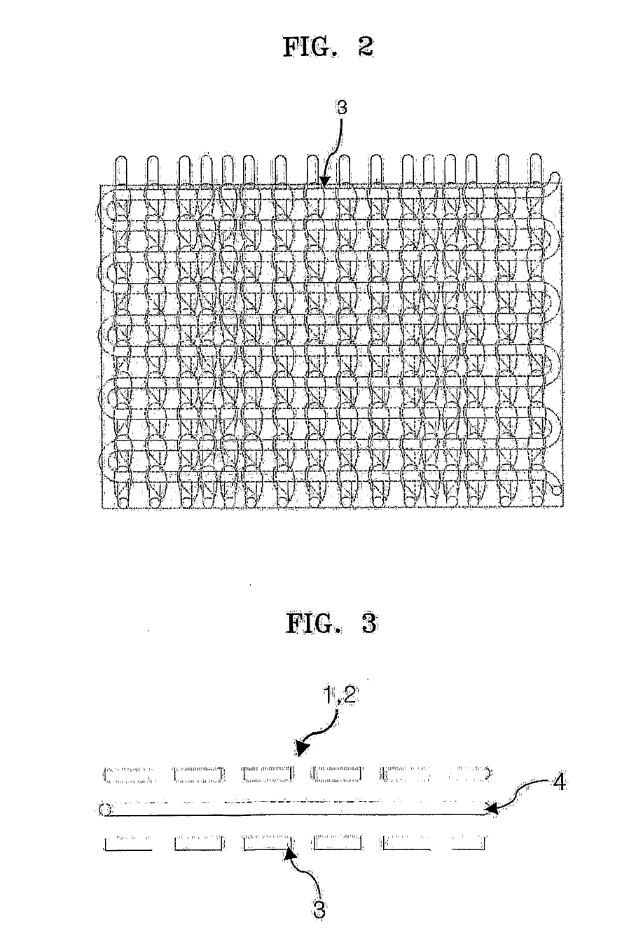

FIG. 2 is a rear view of the conductive elastic band of FIG. 1.

A rear surface 3 of the conductive elastic band is weaved with the general fiber to have non-conductivity. The fabric is weaved using a chain stitch for fixing both surfaces (front and rear surfaces) simultaneously.

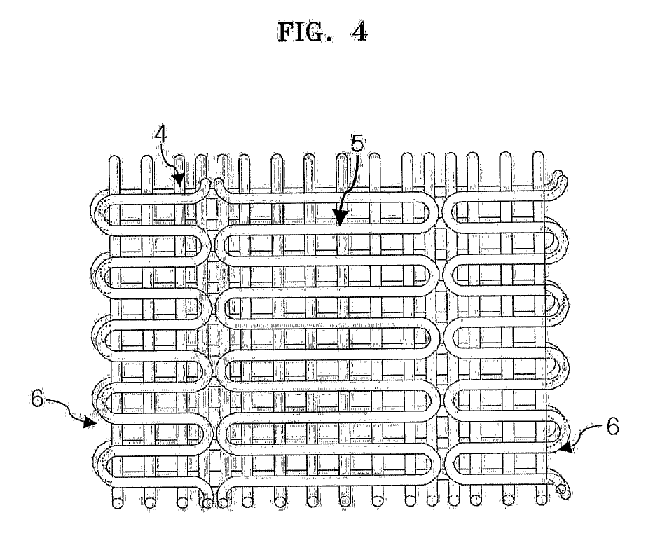

FIG. 3 is a simplified side view of the conductive elastic band of FIG. 1.

A warp 4 is located between the front surface of the elastic band including the conductive portion 1 that will be used as the fabric electrode and the portion 2 to be sewed with the general fiber and the rear surface 3 of the elastic band. The warp 4 applies elasticity to the band using an elastic rubber thread whose elasticity can vary.

FIG. 4 is a detailed front view showing structural elements of the conductive elastic band of FIG. 1.

Conductive woofs 5 have a width and a thickness that can be freely adjusted according to the shape attached to the user and the size of the sensor, and the non-conductive woofs 6 also can be adjusted in width and thickness thereof.

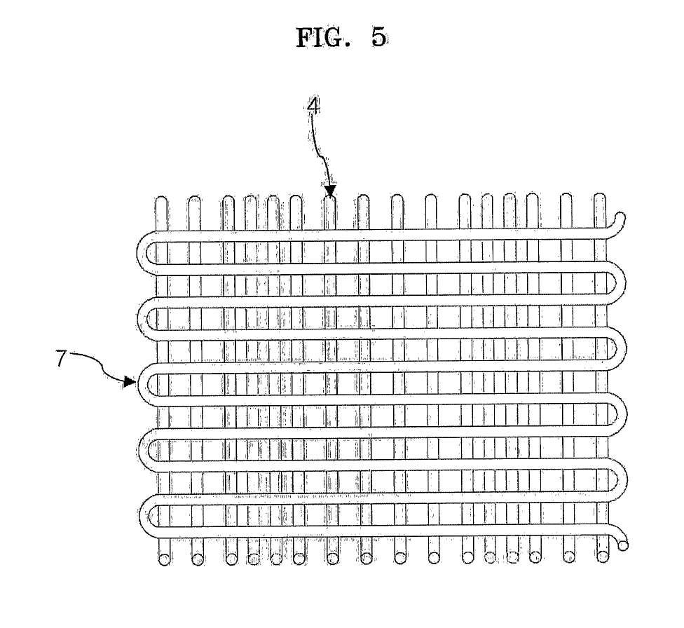

FIG. 5 is a detailed rear view showing structural elements of the conductive elastic band of FIG. 1.

The elastic rubber thread is used for the warp 4 and the woof 7, made of the general fiber, is placed on the warp 4. The woof 7 can be weaved using various fibers, and if necessary, the conductive thread and the general fiber can be used together to form the rear surface like the front surface.

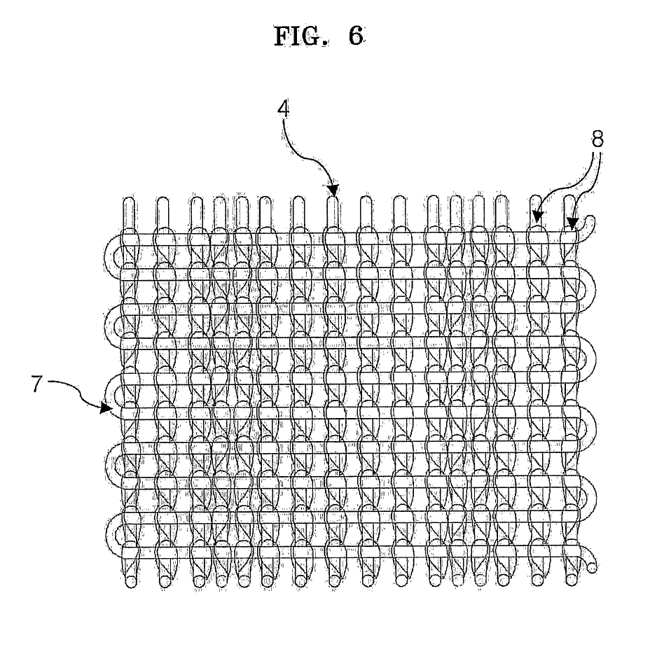

FIG. 6 is a rear view showing structural elements of the conductive elastic band of FIG. 1. The conductive surface and the non-conductive surface are fixed by chain stitching the warp 4 and the general fiber woof 7 using the general fiber 8.

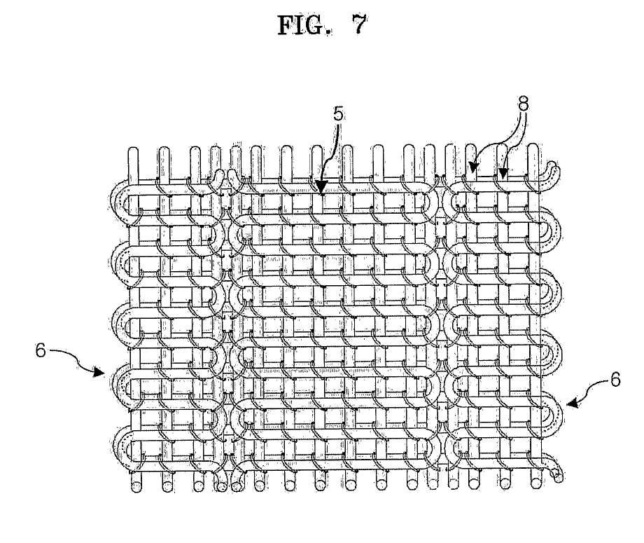

FIG. 7 is a front view showing structural elements of the conductive elastic band of FIG. 1. A conductive portion 5, a non-conductive portion 6, the warp, and the rear surface are fixed altogether by the chain stitching using the general fiber 8.

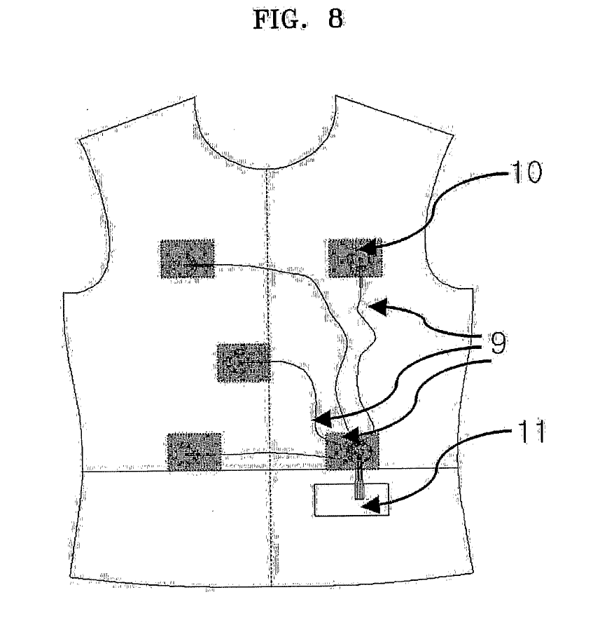

FIG. 8 shows a wearable cloth for obtaining vital information using the conductive elastic band 10 of FIG. 1 and the conductive elastic threads 9 according to an embodiment of the present invention.

Since the conductive elastic band 10 for obtaining vital information such as an electrocardiogram, an electromyogram, or a breathing rate has a superior elasticity, the conductive elastic band 10 can be attached to cloth having high elasticity such as jersey, span, or knit without interfering with the elasticity of the cloth. In addition, the conductive elastic band 10 can make up the cloth attached to the human body, and thus, the vital information can be obtained sufficiently. Here, the conductive elastic band 10 is adhered to the cloth so as to be attached to portions of the human body, from which the vital information will be obtained, and the conductive elastic thread 9 is sewed with the conductive elastic band 10 and connected to a vital information measuring module 11.

The vital information measuring module 11 can include an external communication function, as well as the vital information measurement function, so that the vital information can be transferred to cellular phones or to personal digital assistances (PDAs) according to the user's needs. Thereby, it can provide the user with personal health care information.

The above cloth can maximize the comfort and freedom of movement for the user, and can remain attached to the user so as to obtain the vital information stably.

As described above, the conductive elastic band of the present invention can ensure the comfort of the user when it is applied to cloth, and can obtain the vital information in various actions of the user. The shape of the conductive elastic band can be changed according to the object, and the elasticity can be adjusted also.

In addition, the vital information obtained can be transferred using the conductive elastic band and the conductive thread without using an additional connection method, and the conductive elastic band and thread can be imbedded in the cloth, and thus, the comfort of the user can be improved. The vital information obtaining cloth that provides the user with comfort and freedom of movement can be worn by soldiers, fire fighters, operators working in dangerous environment, elderly people, and people having cardiovascular disorders for monitoring their vital information continuously. Thereby, accidents can be dealt with rapidly.

According to the present invention, the area of the conductive sensor in the conductive elastic band can be freely adjusted according to the object of using the sensor, and the sensor can be stably attached to the human body due to the high elasticity while maintaining a level of comfort for the user. The information obtained using the conductive elastic band is transferred through the conductive elastic threads without interfering with the actions of the human body, and thus, the vital information can be obtained stably while increasing the freedom and the comfort of the user. Moreover, the conductive elastic band can obtain and transfer the vital information in all kinds of clothes such as functional sports wears, outdoor clothes, patient clothes, and specialized functional wears.

In particular, the conductive elastic band of the present invention is weaved using different kinds of fibers and structures on the front and rear surfaces so as to have the conductive surface and the non-conductive surface, and thus, it can be efficiently used to obtain the vital information of the user. In addition, the conductive elastic band can be washed, managed, and formed in various shapes in bio-clothes obtaining the vital information while maintaining the comfort of the user, and thus, can solve the problems of the conventional non-flexible wires or non-elastic conductive threads.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A conductive elastic band comprising:
a front surface in contact with the a human body and including a conductive portion having a fabric electrode and a non-conductive portion weaved with general fibers;
a non-conductive rear surface weaved with the general fibers; and
elastic warps located between the front surface and the rear surface.

2. The conductive elastic band of claim 1, wherein the front surface is weaved using woofs, and the woofs include conductive woofs on the conductive portion that can sense vital signals from the human body and non-conductive woofs on the non-conductive portion that can be sewed with the general fibers.

3. The conductive elastic band of claim 2, wherein the warps are elastic rubber threads providing elasticity to the conductive elastic band.

4. The conductive elastic band of claim 3, wherein the warps and the woofs are stitched by a chain stitching to fix the front surface and the rear surface simultaneously.

5. The conductive elastic band of claim 4, wherein the rear surface is weaved using non-conductive woofs.

6. The conductive elastic band of claim 5, wherein the non-conductive woofs of the rear surface and the warps are stitched using the chain stitching so as to fix the front surface and the rear surface simultaneously.

7. The conductive elastic band of claim 6, wherein conductive elastic threads are sewed and connected to the conductive elastic band to transfer the user's vital signals externally.
